(19) United States
(12) Patent Application Publication
(10) Pub. No.: US 2004/0029137 A1
(43) Pub. Date: Feb. 12, 2004

(54) Secure optically readable adhesive or coating liquid product, method for marking products with a liquid ink and uses of said product

(76) Inventor: Sebastien De Lamberterie, Paris (FR)

Correspondence Address: Allan M Lowe, Lowe Hauptman Gopstein Gilman & Berner, 1700 Diagonal Road, Suite 310, Alexandria, VA, 22314, US

(21) Appl. No.: 10/343,123

(22) PCT Filed: Jul. 27, 2001
(86) PCT No.: PCT/FR01/02480

(30) Foreign Application Priority Data
Jul. 28, 2000 (FR) 00 09948

(51) Int. Cl. C12Q 1/68; G01N 33/53

(52) U.S. Cl. 435/6; 435/7.1

(57) ABSTRACT

The invention concerns a secure optically readable adhesive or coating liquid product, characterised in that it comprises a minimal proportion of fluorescent molecules, invisible in daylight after its application on the support, but optically readable by epifluorescence in an excitation wavelength range included in the visible.

The invention relates to an optically detectable secure adherent or coating liquid product, a product marking method using a liquid ink and uses of such a product.

The marking of products, such as banknotes, using inks or coatings comprising optically detectable fluorescent elements is known in the prior art. The fluorescence of the inks is detected by UV radiation, the spectrum of which is between approximately 0.01 μm and 0.385 μm, or by off-center UV radiation, or by IR radiation, the spectrum of which is between 0.76 μm and 1 mm, or by grazing light type radiation.

An ink invisible to the naked eye marketed by the Eastman Chemical Company under the trademark Clir Code is also known. This ink can only be detected by a specific device allowing to detect fluorescence close to the infrared range between 0.7 μm and 1 mm.

In addition, tracing products by means of molecular tracers, such as is DNA strands, the presence of which is detected for example by fluorescent spherical particles is known; these tracers are known through the following patents: EP 0 527 850, EP 0 657 028, EP 94 920 549.6. The spherical particles are bound to fluorescent molecules detectable by epifluorescence microscopy. When the spherical particles are detected by fluorescence, the DNA strands which were deposited with the fluorescent particles, are detected and can be taken.

A first aim of the invention is to propose an adherent or coating liquid product ensuring secure marking, undetectable to the naked eye, of the item to which it is applied. This will enable the secret traceability or authentication of the marked item.

This aim is achieved by the optically detectable secure adherent or coating liquid product, said product being characterised in that it comprises a minimal proportion of fluorescent molecules, invisible in daylight after application on the base, but optically detectable by epifluorescence in an excitation wavelength range comprised in the visible range.

Other characteristics of the optically detectable secure ink according to the invention are defined below.

Another aim of the invention is to propose a product marking method using an optically detectable ink.

This aim is achieved by the product marking method using an optically detectable liquid ink which comprises a deposition step on the surface of the product to be marked of a small quantity of secure ink, detectable by epifluorescence, in an excitation wavelength range comprised in the visible range, to form a printed code.

Other characteristics of the product marking method according to the invention are defined below.

The invention also relates to the use of the product to authenticate, by marking, works of art, textiles, hollow or flat glass (by surface treatment, serigraphy, or ink jet printing), metal spare parts (by surface treatment), aluminium blisters (by photogravure or flexographic printing), security papers, banknotes and sealing papers (tax or works of art).

Other embodiments and advantages of the present invention will be seen more clearly upon reading the following disclosure with reference to the appended figures wherein:

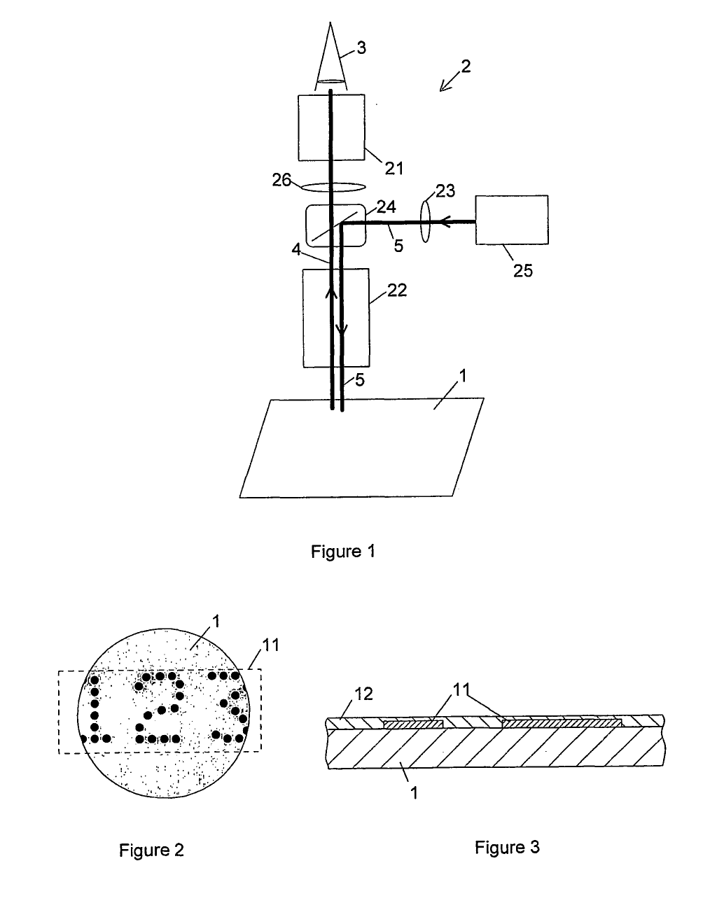

FIG. 1 schematically represents the observation by epifluorescence microscopy of a product marked using the marking method according to the invention;

FIG. 2 represents the field of vision in the microscope eyepiece of the marking of a product;

FIG. 3 represents a sectional view of a base marked using the marking method according to the invention;

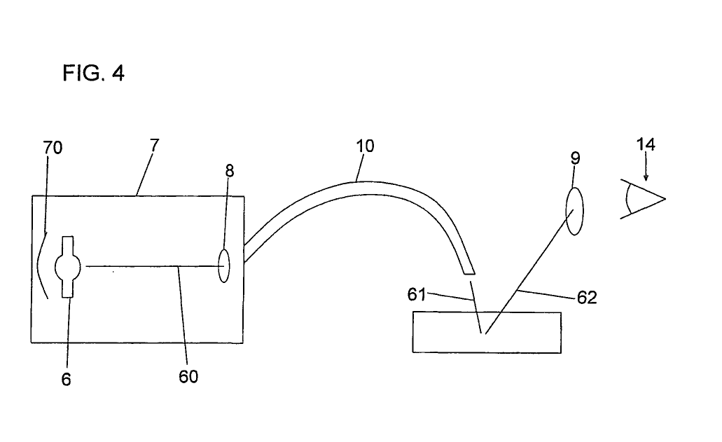

FIG. 4 schematically represents the observation by epifluorescence using a different device, of a product on which the product according to the invention has been applied.

In the disclosure hereafter, the term adherent or coating liquid product refers to any type of product that can be applied to a base. The adherent or coating liquid product may be for example an adhesive, an ink, a varnish, a lacquer, an oil, such as an oil for metal parts or used for lubricating textile yarns, a surface agent having scratch-proof, adhesion-proof, etc. properties.

The adherent or coating liquid product according to the invention comprises fluorescent molecules, detectable by epifluorescence. Episcopy is distinguished from diascopy in that, in the former, the excitation radiation of the observed object does not pass through said object, while in diascopy, the light source is on the other side of the observed object with respect to the observer.

The fluorescent molecules according to the invention are invisible on the marked product to the naked eye or by any other conventional means using, as in the field of secure inks, UV, IR or grazing light type radiation. The fluorescent molecules according to the invention are detected by epifluorescence by being excited by a specified wavelength range of the emission spectrum of a light source. This light source may be for example that integrated in an epifluorescence microscope. Similarly, the excitation of fluorescent molecules, enabling fluorescent emission in a wavelength range between approximately 0.385 and 0.7 μm belonging to the range of radiation visible to the human eye, is only carried out for a determined excitation light intensity, for example between 150 and 300 Candela. The light source may consist, for example, of a mercury, xenon or argon vapour lamp. For example, the fluorescent molecules used in the present invention may consist of fluorescent molecules conventionally used to label spherical particles, DNA strands or proteins, such as those marketed by the company “Molecular Probes”, for example under the references “Alexa Fluor”, “Texas Red”, etc. for aqueous solutions and “BODIPY” for solvent-based solutions (alcohol, acetone, ester, toluene, etc.). For example also, the light spectrum of the mercury vapour lamp selected notably has emission peaks in a wavelength from 0.385 to 0.6 μm and in particular for the following wavelengths: 0.436, 0.546, 0.577-0.579.

According to the invention, the adherent or coating liquid product may be an optically detectable secure ink. Hereafter in the disclosure, the term ink refers to any liquid or pasty preparation used for a printing system. For example, the term ink is used to refer to a coloured or uncoloured ink, or a lacquer or a varnish.

The ink according to the invention comprises fluorescent molecules detectable by epifluorescence. This detection is carried out, for example, using an epifluorescence microscope comprising a light source emitting in the visible range, combined with a set of filters to induce the excitation of the fluorescent molecules contained in the ink around a determined wavelength. The fluorescent molecules are added to the ink at a very low concentration so as not to be visible in daylight on the marked product and in a minimal concentration below which it becomes difficult to detect them. This minimal concentration will be for example 0.001 g/l. If the ink is colourless, the ink base is composed of a liquid mixture of a colourless resin, conventionally used in the field of printing, in a colourless solvent. Depending on the nature of the fluorescent molecules, the ink may be slightly tinted, in solution, before use, but remains totally invisible to the naked eye and in natural light after its application onto the product to be marked.

Behavioural additives, also colourless but affecting for example the viscosity or other physicochemical parameters of the ink, may be added. The fluorescent molecules must have a good solubility in the solvent used to ensure good dispersion of the fluorescent molecules in the solvent.

The product marking method consists of printing on the surface of the product, by means of the invisible ink described above, an identification mark, such as a goldsmith's stamp, or a code, for example alphanumeric. The products may also be marked with an incremented alphanumeric code used to trace each product secretly. In FIG. 1, the visual observation of the code is performed by means of detection means consisting, for example, of an epifluoresence microscope 2.

The epifluorescence microscope comprises an eyepiece 21, for example of a magnification factor of 10, and a lens 22, for example of a magnification factor of 5, between which is placed a dichroic mirror 24. The mirror 24 makes it possible to reflect the radiation from a light source 25 in the direction of the product 1 forming the base, in order to illuminate the marked product 1 from above. The marked product 1 is arranged under the lens 22 and can be observed visually using the eyepiece 21 as represented by the reference 3. The microscope comprises a set of filters 23, 26 adapted to the characteristics of the fluorescent molecule used and of the light source and to the surface of the marked product. This set of filters makes it possible to isolate the photons of the fluorescent emission 4 from the excitation 5 photons emitted by the light source and reflected by the mirror 24 and also to obtain a sufficient contrast between the code and the product. The set of filters comprises one filter 23, so-called the excitation filter, placed between the source 25 and the mirror 24, and one filter 26, so-called the stopping or emission filter, placed between the mirror 24 and the eyepiece 21. The excitation filter 23 will favour the passage of specific wavelengths of the radiation from the selected light source emitting in the visible range. These wavelengths will be determined according to the fluorochrome, i.e. the fluorescent substance selected to produce in a given excitation wavelength in the visible range, a fluorescent emission wavelength in the spectrum of visible radiation. The dichroic mirror is adapted to two excitation and emission wavelength spectra. The stopping filter only allows one or more ranges in the selected wavelength range between 0.4 μm and 0.7 μm to pass. This stopping or emission filter 26 is very important since it makes it possible to:

prevent the passage of the transmitted waves, i.e. not reflected by the dichroic mirror 24, produced by the reflection of incident light on the marked product 1 and directly from the light source and,

select the emission waves emitted by the fluorescence.

The ink, according to the invention, may be deposited in small quantities on the product to be marked using different printing methods, such as serigraphy, photogravure, ink jet, an offset method, flexographic printing or stamp or typographic printing, steel plate printing or heat transfer. The ink may also be deposited manually onto the product by means of a stamp or a pen. In the case of code printing using an ink jet type method, the printing heads enable the formation of small characters and are configured to deposit the least ink possible in order to guarantee the invisibility of the marking. The printing may be inverted on the base in order to take into account the inversion of the view of the printing through the lens. FIG. 2 represents the field of vision at the eyepiece 21 of the code 11 printed on the product 1, with a magnification factor of 50. For example, the code 11 is composed of a set of characters 2 millimetres high.

The proportion of fluorescent molecules in the ink used must be sufficiently high to enable the printing of an easily detectable code, by the epifluorescence equipment, while remaining invisible in daylight. In this way, at the present time, with the equipment available in terms of lamp power, lamp type and optical efficiency, for example a minimum of the order of 0.001 g/l is used. The proportion of fluorescent molecules in the ink may have an upper limit according to the solubility of the fluorescent molecules in the solvent and/or according to the printing method. In this case, this upper limit will be observed.

The ink, according to the invention, may be deposited on any type of object such as flasks, bottles, for example made of glass or plastic, or packagings, for example made of cardboard, to mark or identify the products they contain. The ink, applied for example manually, for example with a felt pen or a stamp, may also be used to trace or authenticate important objects or documents such as contracts, invoices, wills, works of art.

With reference to FIG. 3, one or more layers 12, so-called the coating layers, are deposited on the printed code 11. Said coating layer covers at least part of the surface of the product 1 comprising the printed code 11 and may, depending on the application, cover the entire surface of the product 1. Said coating layer 12 makes it possible to render the printed code totally invisible to the naked eye, even with grazing light. Indeed, the layer of ink forming the code 11, even free from pigments or dyes, may be visible, either due to the thickness of the ink on the base, or due to etching by certain solvents in the ink or due to a gloss effect. Said coating layer provides a homogenisation of the surface of the product at the place where the code is printed and a physical protection of the printed code by preventing the removal of the code. The coating layer may be obtained by depositing a layer of varnish, by means of a film-coating operation using for example a plastic film, or by depositing a layer of non-opacifying ink for the fluorescence of the ink used to form the printed code. In this way, in the case of a secure code on a packaging, for example, the code may be deposited by ink jet and coated with a coating layer, for example such as serigraphic ink or offset ink. In order to remove such a marking, it is necessary to dissolve the coating layer before dissolving the code, thus causing irreversible damage to the packaging.

The total invisibility of the printing with the ink, according to the invention, may be obtained without requiring a coating layer when the product has a porous surface and the ink can impregnate the product.

In the case of printing of the code on cardboards or other porous bases, printed or not, such as bottle labels or the reverse side of packaging such as perfume boxes, the secure code may be deposited directly onto the finished product and remains totally invisible, without requiring a coating layer due to the porosity of the cardboard or the base. In the case of cardboard packagings, so-called the closed cardboard packagings, comprising a varnishing or film-coating such as most of the outer surfaces of packagings, the marking may be performed before the film-coating or the varnishing. On some non-film-coating and non-varnished closed surfaces, the code may be deposited under a coating layer, of the offset or serigraphic type for example, not preventing the optical detection of the secure marking.

Printing small characters with a small quantity of ink makes it possible to position the code on or under non-modifiable text on the packaging such as the name, trademark, contents or product bar code.

In order to ensure that the ink is totally tamper-evident and dedicate a single ink to an application, the ink may comprise tracer molecules such as DNA strands and/or fluorescent microspheres. The inks may be customised by selecting the fluorescent molecule and the DNA code. The marking is detected by direct observation of the surface of the product, for example by epifluorescence microscopy, with no destruction of or damage to the surface. After the detection of the marking by epifluorescence, the marking content authentication operation is performed by reading the DNA code which makes it possible to ensure the marking is tamper-evident and to ensure traceability, but causes, due to the removal of the ink, damage to or destruction of the surface. In this way, it is possible to envisage replacing the DNA tracer by any other type of tracer such as for example colorimetric tracers changing colour by chemical addition with another product, or chemiluminescent additives marketed by the company M.L.T. and disclosed in the American patent U.S. Pat. No. 5,879,946.

According to the invention, the adherent or coating liquid product may also be applied by heat transfer onto the marking base, for example labels or cards. This operation will be conducted by coating a heat transfer cylinder with the product according to the invention.

According to the invention, another detection system can be used to observe the fluorescence by means of epifluorescence without using a microscope. For this, with reference to FIG. 4, this detection system comprises a light source 6 placed in a drilled box 7, consisting, for example, of a 100 Watt mercury vapour lamp and equipped with a reflector 70 to reflect the light to the drilling produced in the box 7. According to the invention, this light source 6 emits in the visible range. As for the epifluorescence microscope, this detection system comprises a set of filters adapted to the characteristics of the fluorescent molecule used and of the light source 6 and to the surface of the marked product 13. This set of filters comprises, as for the microscope, one excitation filter 8 placed between the source 6 and the marked object 13, for example in the box 7 in front of the bore and makes it possible to filter the light 60 emitted by the light source 6 so as to obtain one or more determined wavelengths. The light filtered by the excitation filter 8 then passes into a light guide 10 placed at the drilling outlet. This light guide 10 is responsible for conveying the filtered light 61 to the secure marking carried out on an object 13. The set of filters also comprises an emission filter 9 responsible for only allowing the passage, among the wavelengths of the light 62 emitted by the fluorescent molecules, of one or more wavelength ranges in the visible range so that they can be observed with the naked eye 14 or using a camera.

In this new device, the waves emitted by the marked product are waves produced by the reflection of the incident light on the product, those forming the fluorescence background noise on the object and those produced by the emission of the specific fluorescence.

The excitation waves which are reflected on the product form a selected light in the visible range, i.e. a visible light of determined colour. Therefore, this light is reflected by the marked product and prevents the observation of the waves produced by the fluorescence emission. The level of the reflection of the incident light on the marked product depends on the colour and nature of the surface of the illuminated product. When the reflection is low, the waves produced by the fluorescence emission are also attenuated. In a manner known in the prior art, for all the product surface types, the reflection of the waves produced by the source prevents the visibility of the waves produced by the fluorescence emission, in spite of the presence of excitation in a specific wavelength range. Therefore, the emission filter 9 serves to prevent the passage of the waves produced by the reflection on the product and also to improve the contrast by eliminating part of the waves forming the fluorescence background noise of the product.

For the observation by epifluorescence of fluorescences emitting in the UV range, the stopping filter is not necessary. Indeed, since the visibility of the excitation waves is very low, the reflection of the excitation waves does not hinder the observation of the emitted waves produced by the fluorescence.

According to the invention, the adherent or coating liquid product may be an oil used for example for lubricating textiles.

Synthetic textile yarns, for example made of polyamide, are most frequently, after extrusion, lubricated with a mineral oil, which gives them a greater meshing ability for fabric formation. The lubricating consists of depositing a thin layer of oil around a textile yarn.

In practical terms, a reel of unprocessed yarn is unreeled, the unreeled yarn then passes onto an oil wipper roller impregnated with oil and is then reeled again. The weight of the oil deposit on the yam is of the order of 2% of that of the yarn.

By dispersing fluorescent molecules, for example in a proportion of 10 ppm, which are excited in wavelengths in the visible range in lubricating oil, the lubricating oil is visibly coloured. This colouring may be produced in red for example with molecules marketed under the reference “Alexa 568” by the company “Molecular Probes”. After lubrication, the secure marking of the textile yarns lubricated with the oil comprising the fluorescent molecules, is invisible in daylight. This is due to the fact that the lubricating oil is always deposited in very small quantities on textile yams.

The fluorescent marking in the visible range is detected by positioning a yarn lubricated with the marked oil, under the lens of an epifluorescence microscope equipped with filters corresponding to the excitation and emission wavelength ranges of the fluorescent molecules introduced into the oil. In practical terms, the fluorescent marking is identified by comparing an unmarked lubricated yarn, i.e. without fluorescence, with a marked lubricated yarn, i.e. with fluorescence.

It will also be possible to detect the secure marking by exciting the fluorescent molecules directly on a textile produced using lubricated yarns with a specific light, of determined intensity and wavelength, the fluorescence being detected via an emission filter.

Similarly, the adherent or coating liquid product according to the invention may consist of an impregnation solution generally giving textiles certain properties. These solutions may give textiles textural, rigidity, waterproof, mosquito-proof, etc. properties. The impregnation solutions comprising specific fluorescent molecules mark the textiles on which they are applied in a manner that is undetectable in daylight. When the marking is detected, the visibility of the fluorescence emitted from a fabric marked with fluorescence will increase with respect to the lightness of the fabric.

Similarly, fluorescent molecules may be introduced into dye baths, into a glass surface treatment solution, into a solution used for the surface treatment of spare parts.

According to the invention, it will also be possible to use the markings according to the invention for applications other than security. Indeed, it is known in the prior art how to introduce fluorescent products detectable under UV light into varnishes, surface agents, adhesives or other media intended to be deposited onto objects. The purpose of these fluorescences detectable under UV is to be able to monitor the deposition of the medium on the object by observing the fluorescence emitted by excitation under UV light.

According to the invention, to fulfil the same purpose, it will be possible to use fluorescent molecules detectable by excitation in the visible range. Several types of fluorescent molecules excitable at different wavelength ranges may be added to the medium. The excitation will be carried out either separately, i.e. by changing excitation and emission filters for each fluorescent molecule contained in the medium or by exciting all the fluorescent molecules at the same time by using a source filtered by a multipassband, the observation being carried out also via a multipassband filter.

Therefore, it will be possible to add different fluorescent molecules for each layer of medium deposited on the object or different fluorescent molecules for each medium deposited on the object. It will thus be possible to observe the different layers of the medium deposited or intersections between the different media deposited, it being possible to make this observation visually or using a camera using for example the device represented in FIG. 4 and described above.

In this way, the optically detectable secure adherent or coating liquid product is characterised in that it comprises a minimal proportion of fluorescent molecules, invisible in daylight after application onto the base, but optically detectable by epifluorescence in an excitation wavelength range comprised in the visible range.

According to another embodiment, the fluorescent molecules are optically detectable using a light source 6 placed in a drilled box 7, emitting in the visible range to the marked base 13 and an emission filter 9 filtering the specific wavelengths of the fluorescence.

According to another embodiment, the fluorescent molecules are optically detectable using an epifluorescence microscope 2.

According to another embodiment, the epifluorescence microscopy is defined by an excitation lamp 25 with a light intensity between 150 and 300 candela and the use of a set of excitation and emission filters 23, 26 adapted firstly to the characteristics of the light source, secondly to the fluorescent molecule used and also to the marking base 1.

According to another embodiment, the minimal proportion is 0.001 g of fluorescent molecules per litre of solution.

According to another embodiment, the product is an ink.

According to another embodiment, the ink is colourless and comprises a colourless resin and solvent base, rendering it invisible on the base.

According to another embodiment, the ink type is ink jet ink, offset ink, serigraphic ink, photogravure ink, flexographic printing ink, stamp printing ink, typographic or steel plate printing ink.

According to another embodiment, the ink comprises DNA tracer molecules and/or fluorescent microspheres.

According to another embodiment, the solvent type is water, MEC, or ethanol, ester, ester-alcohol mixture, cyclohexane, toluene.

The product marking method using an optically detectable liquid ink is characterised in that it comprises a deposition step on the surface of the product 1 to be marked of a small quantity of secure ink, detectable by epifluorescence in an excitation wavelength range comprised in the visible range, to form a printed code 11.

According to another embodiment, the ink is deposited on a heat transfer cylinder to be deposited on the surface of the product to be marked.

According to another embodiment, the deposition of the secure ink is performed by means of serigraphy, photogravure, ink jet, an offset method, flexographic printing or industrial or manual stamp printing, typographic printing, steel plate printing or felt pen.

According to another embodiment, the printed code 11 is an identification mark.

According to another embodiment, the marked product consists of a porous base.

According to another embodiment, the secure ink deposition step is followed by a deposition step of at least one coating layer 12, not totally opacifying for the fluorescence of the secure ink, on at least the marked part of the surface of the product 1, in order to render the thickness of the secure ink deposition invisible.

According to another embodiment, the coating layer consists of a layer of varnish, a film-coating, or a layer of ink.

The invention also relates to the use of the product to authenticate, by marking, works of art, textiles, hollow or flat glass (by surface treatment, serigraphy, or ink jet printing), metal spare parts (by surface treatment), aluminium blisters (by photogravure or flexographic printing), security papers and banknotes. The term security papers refers to coated papers such as cheques, titles, identity cards, labels, tax strips or work of art sealing papers, etc.

It should be clear to those skilled in the art that the present invention enables embodiments in numerous other specific forms without leaving the scope of the invention as claimed. Consequently, the present embodiments must be considered as an illustration, but may be modified in the field defined by the scope of the claims attached, and the invention must not be restricted to the details given above.

1. Optically detectable secure adherent or coating liquid product, characterised in that it comprises a minimal proportion of fluorescent molecules, invisible in daylight after application on the base, but optically detectable by epifluorescence in an excitation wavelength range comprised in the visible range.

2. Product according to claim 1, characterised in that the fluorescent molecules are optically detectable using a light source (6) placed in a drilled box (7), emitting in the visible range to the marked base (13) and an emission filter (9) filtering the specific wavelengths of the fluorescence.

3. Product according to claim 1, characterised in that the fluorescent molecules are optically detectable using an epifluorescence microscope (2).

4. Product according to claim 3, characterised in that the epifluorescence microscopy is defined by an excitation lamp (25) with a light intensity between 150 and 300 candela and the use of a set of excitation and emission filters (23, 26) adapted firstly to the characteristics of the light source, secondly to the fluorescent molecule used and also to the marking base (1).

5. Product according to any of claims 1 to 4, characterised in that the minimal proportion is 0.001 g of fluorescent molecules per litre of product.

6. Product according to any of claims 1 to 5, characterised in that it is an ink.

7. Product according to claim 6, characterised in that the ink is colourless and comprises a colourless resin and solvent base, rendering it invisible on the base.

8. Product according to claim 6 or 7, characterised in that the ink type is ink jet ink, offset ink, serigraphic ink, photogravure ink, flexographic printing ink, stamp printing, typographic or steel plate printing ink.

9. Product according to any of claims 6 to 8, characterised in that the ink comprises DNA tracer molecules and/or fluorescent microspheres.

10. Product according to any of claims 7 to 9, characterised in that the solvent type is water, MEC, or ethanol, ester, ester-alcohol mixture, cyclohexane, toluene.

11. Product marking method using an optically detectable liquid ink, characterised in that it comprises a deposition step on the surface of the product (1) to be marked of a small quantity of secure ink, detectable by epifluorescence in an excitation wavelength range comprised in the visible range, to form a printed code (11).

12. Product marking method according to claim 11, characterised in that the deposition of the secure ink is performed by means of serigraphy, photogravure, ink jet, an offset method, flexographic printing or industrial or manual stamp printing, typographic printing, steel plate printing or felt pen.

13. Product marking method according to claim 11, characterised in that the ink is deposited on a heat transfer cylinder to be deposited on the surface of the product to be marked.

14. Product marking method according to claim 11 or 12, characterised in that the printed code (11) is an identification mark.

15. Product marking method according to any of claims 11 or 12 or 14, characterised in that the marked product consists of a porous base.

16. Product marking method according to any of claims 11 or 12 or 14 or 15, characterised in that the secure ink deposition step is followed by a deposition step of at least one coating layer (12), not totally opacifying for the fluorescence of the secure ink, on at least the marked part of the surface of the product (1), in order to render the thickness of the secure ink deposition invisible.

17. Product marking method according to claim 16, characterised in that the coating layer consists of a layer of varnish, a film-coating, or a layer of ink.

18. Use of the adherent or coating liquid product according to claims 1 to 10 in the field of works of art.

19. Use of the adherent or coating liquid product according to claims 1 to 10 to mark textiles.

20. Use of the adherent or coating liquid product according to claims 1 to 10 to mark hollow or flat glass by surface treatment, serigraphy or ink jet printing.

21. Use of the adherent or coating liquid product according to claims 1 to 10 to mark metal spare parts by surface treatment.

22. Use of the adherent or coating liquid product according to claims 1 to 10 to mark security papers.

23. Use of the adherent or coating liquid product according to claims 1 to 10 to mark banknotes.

24. Use of the adherent or coating liquid product according to claims 1 to 10 to mark pharmaceutical blisters by photogravure or flexographic printing.
